(19) United States
(12) Patent Application Publication
(10) Pub. No.: US 2008/0221655 A1
(43) Pub. Date: Sep. 11, 2008

(54) Bifurcated Balloon and Stent

(75) Inventor: Amir Miller, Ramat-Hasharon (IL)

Correspondence Address: DARBY & DARBY P.C., P.O. BOX 770, Church Street Station, New York, NY, 10008-0770, US

(73) Assignee: B-BALLOON LTD., Or Yehuda (IL)

(21) Appl. No.: 12/067,599

(22) PCT Filed: Sep. 21, 2006
(86) PCT No.: PCT/IL2006/001111

§ 371 Date: May 7, 2008

Related U.S. Application Data
(60) Provisional application No. 60/719,737, filed on Sep. 21, 2005.
(60) Provisional application No. 60/750,024, filed on Dec. 12, 2005.

(51) Int. Cl. A61F 2/84; A61F 2/82

(52) U.S. Cl. 623/1.11; 623/1.35

(57) ABSTRACT

A stent (22) for the treatment of a vascular bifurcation includes a distal member (40) and a proximal member (42), the distal member and the proximal member comprising tubular bodies, the distal member comprising first struts (64, 66) oriented in the proximal direction and the proximal member including second struts (54, 56) oriented in the distal direction, such that at least one of the first struts is connected to at least one of the second struts on a first side of the stent (46), while on a second side (48) of the stent, opposite the first side, one or more of the first struts are unconnected to any of the second struts and one or more of the second struts are unconnected to any of the first struts.

### CROSS-REFERENCES TO RELATED APPLICATIONS

This application claims the benefit of U.S. Provisional Patent Application 60/719,737, filed Sep. 21, 2005, and of U.S. Provisional Patent Application 60/750,024, filed Dec. 12, 2005, which are incorporated herein by reference. This application is related to PCT Patent Publication WO 2005/041810 A2, filed Nov. 2, 2004, which is assigned to the assignee of the present patent application and which is incorporated herein by reference.

### FIELD OF THE INVENTION

The present invention relates generally to vascular catheterization, and specifically to intravascular balloons and stents.

### BACKGROUND OF THE INVENTION

Intravascular stents are used for various purposes, including opening occluded blood vessels. Typically, the stent is supplied in a narrow, contracted form, with a deflated balloon contained inside the stent. The stent and balloon are held at the distal end of a catheter. The operator inserts a guide wire into the blood vessel, and then slides the catheter over the wire to position the stent in the proper location. The balloon is then inflated, via a channel in the catheter, causing the stent to expand so as to be anchored in place and hold the vessel open. Once the stent has been expanded, the balloon is deflated and is withdrawn, along with the catheter, from the vessel.

A stent may be positioned at the location of a bifurcation, where two blood vessels meet. The abovementioned PCT Patent Publication WO 2005/041810 A2 describes apparatus for treatment of a vascular bifurcation. A balloon configured to be deployed at a bifurcation comprises two parts, a main, longitudinal part and a protrusion. When the balloon is uninflated, the protrusion may be folded against the main part or contained within the main part. Upon partial inflation of the balloon, the protrusion may facilitate alignment of the balloon at the bifurcation site. A stent having a side opening may be fit over the main part and oriented so that the protrusion protrudes through the side opening into a side vessel when the balloon is inflated.

A stent may be positioned to support both a main vessel and a side vessel that diverge from a point of bifurcation. Stents having a Y-shape and configured to be positioned in this manner are known in the art. For example, U.S. Pat. No. 4,994,071 to MacGregor, whose disclosure is incorporated herein by reference, describes a bifurcating stent with first and second matrices of interconnected loops, each matrix defining a separate fluid flow paths. A flexible member connecting the matrices is bent to define an angle between the flow paths. The matrices may be expanded when placed in a subject vessel.

U.S. Pat. No. 6,210,380 to Mauch, whose disclosure is incorporated herein by reference, describes a method for delivering a bifurcated catheter having two branches that carry a Y-shaped stent. A coupler is mounted at the distal ends of the two branches, thereby holding the branches together until the catheter is positioned at the bifurcation site. Two balloons are used to inflate the Y-shaped stent when the catheter is positioned at the bifurcation site, one branch being positioned in the main vessel and the other branch in the side vessel.

U.S. Pat. No. 6,210,429 to Vardi et al., whose disclosure is incorporated herein by reference, describes a method for positioning a stent having a side hole at a vessel bifurcation. The side hole may be surrounded by an expandable portion that is integrally formed with the stent body. During positioning of the stent, the expandable portion is flush with the stent body. The stent is positioned such that the side hole is aligned with an ostium of a branch vessel. The stent is expanded by a first balloon, and the expandable portion is then inflated by a second balloon which extends through the side hole into the branch vessel. Subsequently, a second stent may be positioned in the branch vessel through the side hole.

### SUMMARY OF THE INVENTION

Embodiments of the present invention provide novel methods for treatment of vascular bifurcations, as well as stents, balloons, and ancillary components for use in such treatment. (The term “bifurcation” as used herein refers to the area where two blood vessels meet, and includes an ostium.) These methods permit medical practitioners to position stents with enhanced accuracy and ease.

There is therefore provided, in accordance with an embodiment of the present invention, a stent for treatment of a vascular bifurcation including a distal member and a proximal member, the distal member and the proximal member including tubular bodies, the distal member including first struts oriented in the proximal direction and the proximal member including second struts oriented in the distal direction, such that at least one of the first struts is connected to at least one of the second struts on a first side of the stent, while on a second side of the stent, opposite the first side, one or more of the first struts are unconnected to any of the second struts and one or more of the second struts are unconnected to any of the first struts.

Typically, the unconnected first and second struts overlap while the stent is in an unexpanded, linear shape.

The first side generally includes a joint and the stent is operative to bend along the joint, away from the second side, when the stent is moved from a main vessel into a side vessel at the vascular bifurcation. The unconnected first struts may include a constriction of the unconnected second struts, and the unconnected second struts may be forced by the constriction to bend towards the first side responsively to the stent being moved into the side vessel. In some embodiments, the unconnected first struts are operative to release the constriction of the unconnected second struts responsively to further bending of the stent. Alternatively or additionally, the unconnected first struts are operative to release the constriction of the unconnected second struts responsively to an expansion of the stent.

Responsively to being moved into the side vessel, the stent is generally operative to perform an expansion causing the unconnected second struts to support a wall of the main vessel opposite the side vessel and the unconnected first struts to support a wall of the main vessel distal to the bifurcation. In some embodiments, the stent is operative to expand by a balloon that includes a main body and a protrusion, the main body being shaped when expanded to bend within the bifurcation, and the protrusion being shaped when expanded to protrude along the axis of the main vessel and to extend into a portion of the main vessel distal to the bifurcation. The protrusion may be operative to bend the unconnected first struts against the wall of the main vessel distal to the bifurcation.

There is further provided, in accordance with an embodiment of the present invention, a method for manufacturing a stent for treatment of a vascular bifurcation, including:

providing distal and proximal members including tubular bodies, the distal member including first struts oriented in the proximal direction and the proximal member including second struts oriented in the distal direction; and

connecting at least one of the first struts to at least one of the second struts on a first side of the stent, while not connecting one or more of the first struts on a second side of the stent, opposite the first side, to any of the second struts and not connecting one or more of the second struts on the second side to any of the first struts.

In some embodiments, the one or more of the unconnected first struts on the second side overlap the one or more of the unconnected second struts on the second side.

There is further provided, in accordance with an embodiment of the present invention, a method for treatment of a vascular bifurcation having a side vessel branching from a main vessel, the method including:

introducing into the main vessel in proximity to the side vessel a stent including a distal member and a proximal member, the distal member and the proximal member including tubular bodies, the distal member including first struts oriented in the proximal direction and the proximal member including second struts oriented in the distal direction, wherein at least one of the first struts is connected to at least one of the second struts on a first side of the stent, while on a second side of the stent, opposite the first side, one or more of the first struts are unconnected to any of the second struts and one or more of the second struts are unconnected to any of the first struts;

advancing the stent into the side vessel, such that the first side of the stent is bent with the distal member partially within the side vessel, and the second side of the stent faces away from the side vessel;

expanding the stent to support portions of the main vessel and the side vessel; and

bending the unconnected first struts to support a further portion of the main vessel distal to the vascular bifurcation.

In some embodiments, supporting the further portion of the main vessel distal to the vascular bifurcation includes supporting a portion of an ostium of the side vessel.

The method for treatment may include carrying the stent on a balloon having a main body and a protrusion, wherein expanding the stent includes expanding the main body to an expanded shape that bends within the bifurcation, and bending the unconnected first struts includes expanding the protrusion along the axis of the main vessel distal to the bifurcation.

There is further provided, in accordance with an embodiment of the present invention, a method for treatment of a vascular bifurcation having a side vessel branching away from an axis of a main vessel, the method including:

introducing into the main vessel in proximity to the side vessel a balloon having a main body and a protrusion;

advancing the main body partially into the side vessel such that the protrusion faces into a portion of the main vessel distal to the bifurcation;

expanding the main body, causing the main body to assume an expanded shape that bends within the bifurcation; and

expanding the protrusion, causing the protrusion to extend along the axis of the main vessel into a portion of the main vessel distal to the bifurcation.

In some embodiments, the main body carries a stent. The stent may include a distal member and a proximal member, the distal and proximal members including tubular bodies, the distal member including first struts oriented in the proximal direction and the proximal member including second struts oriented in the distal direction, such that at least one of the first struts is connected to at least one of the second struts on a first side of the stent, while on a second side of the stent, opposite the first side, one or more of the first struts are unconnected to any of the second struts and one or more of the second struts are unconnected to any of the first struts. Carrying the stent includes orienting the protrusion to face the second side of the stent, Advancing the main body partially into the side vessel includes advancing the stent into the side vessel, such that the first side of the stent is bent, with the distal member partially within the side vessel and the second side facing away from the side vessel. Expanding the main body includes expanding the stent to support portions of the main vessel and the side vessel. Expanding the protrusion includes bending the unconnected first struts against a wall of the main vessel distal to the bifurcation.

The present invention will be more fully understood from the following detailed description of the embodiments thereof, taken together with the drawings in which:

### BRIEF DESCRIPTION OF THE DRAWINGS

FIGS. 1A-1C are pictorial illustrations of a system for treating a vascular bifurcation, in accordance with an embodiment of the present invention;

FIGS. 2A-2F are pictorial and schematic illustrations of elements of a stent designed to treat a vascular bifurcation, in accordance with an embodiment of the present invention;

FIGS. 3A and 3B are pictorial illustrations of a stent at a vascular bifurcation, in accordance with an embodiment of the present invention; and

FIGS. 4A-4C are pictorial illustrations of a further system for treating a vascular bifurcation, in accordance with an embodiment of the present invention.

### DETAILED DESCRIPTION OF EMBODIMENTS

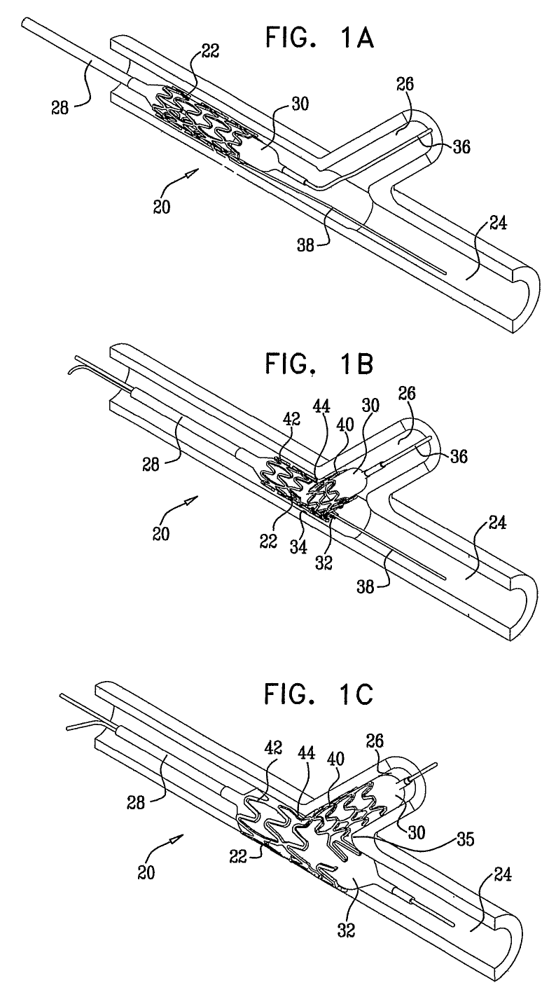

Reference is now made to FIGS. 1A-1C, which are pictorial illustrations of stages of operation of a system 20 for treating a vascular bifurcation, in accordance with an embodiment of the present invention. FIG. 1A is a pictorial illustration of a stent 22 inserted into a blood vessel, referred to hereinbelow as a main vessel 24. Stent 22 is typically crimped on a balloon 30, which is attached to the distal end of a catheter 28. Typically, balloon 30 is configured as an inflatable lumen. Alternatively, or additionally, balloon 30, or particular segments of balloon 30 such as a protrusion 32 described further hereinbelow (FIG. 1B), are configured to be expanded by mechanical means, such as internal springs.

Catheter 28 runs over two guidewires, a guidewire 36 and a guidewire 38, which guide the placement of stent 22 to the location of a vascular bifurcation. Hereinbelow, the vascular bifurcation is understood to be the bifurcation of main vessel 24 and a side vessel 26. Typically, the main vessel follows a generally linear axis past the bifurcation, and the side vessel branches away from that axis.

Guidewire 36 leads from the main vessel into side vessel 26. Catheter 28 is positioned on guidewire 36 such that guidewire 36 extends through the length of balloon 30. Hereinbelow, the length of balloon 30 that slides over guidewire 36 is referred to as the main body of balloon 30.

Guidewire 38 leads through main vessel 24 past the bifurcation. Catheter 28 is positioned on guidewire 38 such that guidewire 38 exits balloon 30 through protrusion 32, which is comprised on a side of balloon 30. Protrusion 32 is an expandable segment of balloon 30 that extends away from the main body when balloon 30 is inflated.

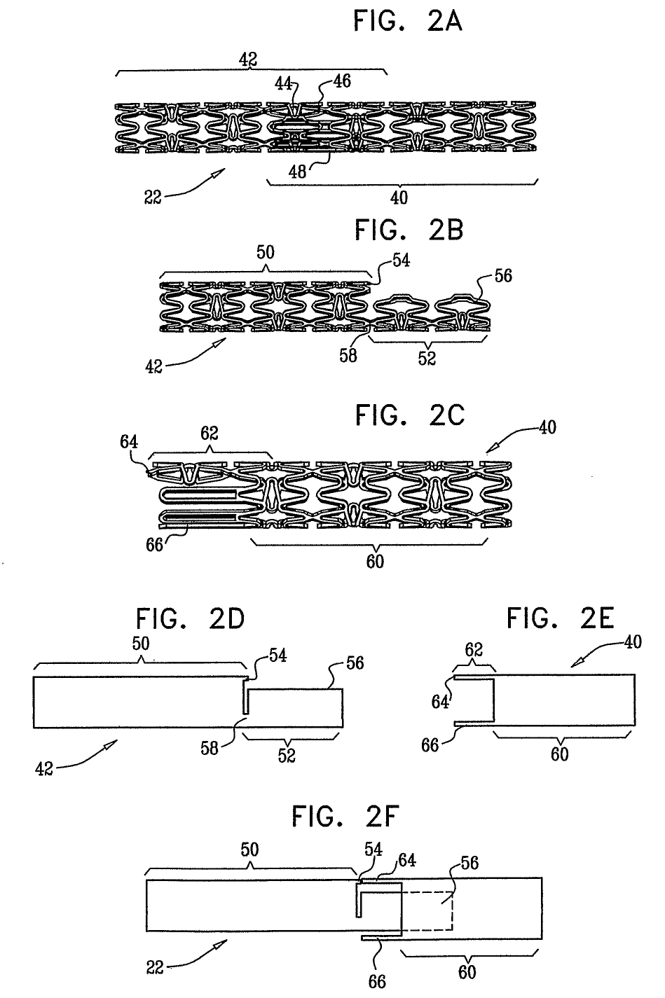

FIG. 1B is a pictorial illustration of stent 22 of system 20 positioned at the vascular bifurcation, in accordance with an embodiment of the present invention. Stent 22 comprises two members, a distal member 40 and a proximal member 42. Positioning stent 22 at the vascular bifurcation comprises moving catheter 28 until distal member 40 of the stent is within the side vessel, while proximal member 42 is in main vessel 24. Moving the stent into the side vessel causes the stent to bend at a juncture, referred to hereinbelow as a joint 44, which connects the distal and proximal members. Joint 44 is comprised of one or more struts of stent 22, as described further hereinbelow (FIGS. 2A-2E). The bending of stent 22 causes protrusion 32 to be aligned along the axis of main vessel 24, facing the extension of main vessel 24 distal to the bifurcation. Bending also positions a distal portion of proximal member 42, described further hereinbelow (FIG. 2B), in proximity to a wall section 34 of the main vessel, opposite the opening of side vessel 26.

FIG. 1C is a pictorial illustration of stent 22 expanded to support portions of the vascular bifurcation, in accordance with an embodiment of the present invention. The main body of balloon 30 may be manufactured to inflate into a curved shape, which may be customized according to the angle at which side vessel 26 branches away from main vessel 24. Inflation of balloon 30 also expands protrusion 32, which emerges into a portion of the main vessel distal to the bifurcation.

Balloon 30 is positioned within stent 22 such that the concave side of the balloon's inflated curvature is positioned against joint 44. Positioning balloon 30 with the curvature towards the joint side also positions protrusion 22 towards the side of the stent opposite the joint (FIG. 2A). When stent 22 is fully expanded, struts of stent 22 may support several portions of the vasculature in the proximity of the bifurcation, including: walls of the main vessel proximal the bifurcation, referred to hereinabove as wall section 34 (FIG. 1B), walls of the side vessel distal to the bifurcation, and the ostium of the side vessel, including the distal portion of the ostium, indicated as a wall section 35 of the bifurcation.

FIGS. 2A-2F are pictorial and schematic illustrations of stent 22 comprising distal member 40 and proximal member 42, in accordance with an embodiment of the present invention. FIG. 2A is a pictorial illustration of stent 22 before the stent is positioned at the vascular bifurcation. Stent 22 has a linear, tubular shape and comprises proximal member 42 and distal member 40. Joint 44 connects the two members on a joint side 46 of stent 22. On the opposite side of the stent, side 48, struts extending from the proximal and distal members overlap, as described further, hereinbelow.

FIGS. 2B and 2C illustrate proximal member 42 and distal member 40, respectively, as they would appear if stent 22 were split at joint 44. It is to be understood that in the application of stent 22, members 40 and 42 are not disassociated. Furthermore, although manufacture of stent 22 may comprise connecting two or more distinct segments (for example, by welding members 40 and 42 at joint 44, or by interlinking struts of the distinct segments), the present invention is not limited to embodiments of stents manufactured from two or more distinct members.

FIG. 2B is an illustration of proximal member 42, which comprises a tubular main section 50. Member 42 is also shown in FIG. 2D, described below. Main section 50 typically comprises a matrix of interconnected or interlaced struts, according to methods of stent manufacture known in the art. At the distal end 52 of proximal member 42, independent sets of struts 54 and 56 extend from main section 50 oriented in the distal direction. Struts 54, on the joint side of stent 22, connect to struts on distal member 40. Struts 56, on the opposite side of stent 22, side 48 (FIG. 2A), are unconnected.

FIG. 2C is an illustration of distal member 40, which comprises a tubular main section 60. Member 40 is also shown in FIG. 2E, described below. Main section 60 typically comprises a matrix of interconnected or interlaced struts. At a proximal end 62 of distal member 40, independent sets of struts 64 and 66 extend from the main section in the proximal direction. Struts 64 are connected to struts 54 of proximal member 42 at joint 44, to form joint side 46 of the stent. It is to be understood that stent 22 may be configured to bend sharply at joint 44, or to bend more gradually along all or a portion of joint side 46. Struts 66 and struts 56 are not connected and instead overlap to form side 48 of stent 22.

FIGS. 2D and 2E are respective schematic illustrations of proximal member 42 and distal member 40. Tubular main sections 50 and 60 are indicated as large blocks. In FIG. 2D, struts 56 are indicated by a thinner block at distal end 52 of proximal member 42. Struts 56 may comprise a partially tubular section that can support part of the inner circumference of a vessel. One or more links 58 connect struts 56 to section 50, thereby enabling distal end 52 of the proximal member to bend inwards, towards joint 44, when stent 22 is bent. In FIG. 2E, struts 64 and 66 are shown extending from main section 60 of member 40 in the proximal direction.

FIG. 2F is a schematic illustration of stent 22, wherein the block illustrations of proximal member 42 and distal member 40 are shown joined together in a complete configuration of stent 22, as represented pictorially in FIG. 1A. FIG. 2F indicates the manner in which proximal struts 66 of distal member 40 overlap distal struts 56 of proximal member 42. Struts 56 are generally positioned internally relative to Struts 66, such that struts 56 extend into section 60. In alternative embodiments, struts 54, 64, 56, and 66 may be configured to be longer or shorter than indicated in FIG. 2F. The relative lengths of these struts determines the overlap of the proximal and distal members on side 48 of stent 22. For example, while struts 56 are indicated as being longer than the total length of struts 54 and 64, such that struts 56 extend into section 60, struts 56 may be relatively shorter and not extend into section 60. Conversely, struts 54 and 64 may be much shorter, such that struts 56 are completely contained within section 60. Struts 66 may also be longer than shown, such that they overlap a portion of section 50.

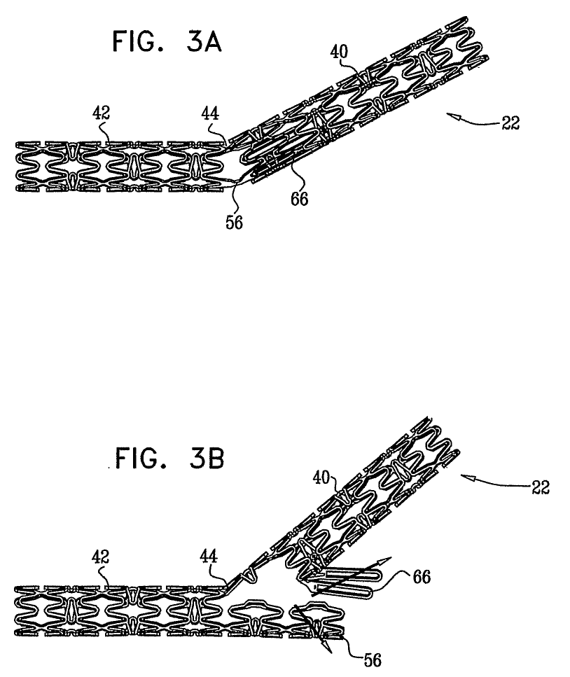

FIGS. 3A and 3B are pictorial illustrations of stent 22 when the stent is positioned at the bifurcation of main vessel 24 and side vessel 26, in accordance with an embodiment of the present invention. FIG. 3A is a pictorial illustration of stent 22 upon being moved initially into side vessel 26. As described in FIG. 1B, hereinabove, distal member 40 moves into the side vessel, causing stent 22 to bend at joint 44. Initial bending of stent 22 on joint side 46 also causes side 48 to bend towards the side vessel at links 58 (FIG. 2D). Struts 56 and 66 remain in an overlapping configuration on side 48 of the stent, as struts 56 confine struts 66 within the circumference of stent 22.

FIG. 3B is a pictorial illustration of stent 22 positioned at the bifurcation, following release of struts 56 from the confines of struts 66. Partial expansion by balloon 30, including expansion due to the inflation of protrusion 32, may cause struts 56 to be released from the confines of struts 66 and straighten towards the distal direction of the main vessel. Alternatively, struts 56 may be released due to further bending of stent 22 in side vessel 26.

Following the release of struts 56, further pressure by protrusion 32 against struts 66 causes struts 66 to bend back towards the distal direction of the main vessel.

As indicated with respect to FIG. 1C, hereinabove, further inflation of balloon 30 causes stent 22 to open against the walls of the main and side vessels. Struts 56 expand to support section 34 of the main vessel wall opposite the bifurcation (FIG. 1B). Depending on the length of struts 56, these struts may also support a portion of the main vessel wall distal to section 34. Expansion of stent 22 also pushes struts 66 against the distal portion of the side vessel ostium, indicated in FIG. 1C as wall section 35. Depending on the length of struts 66, these struts may also support a portion of the main vessel wall distal to section 35.

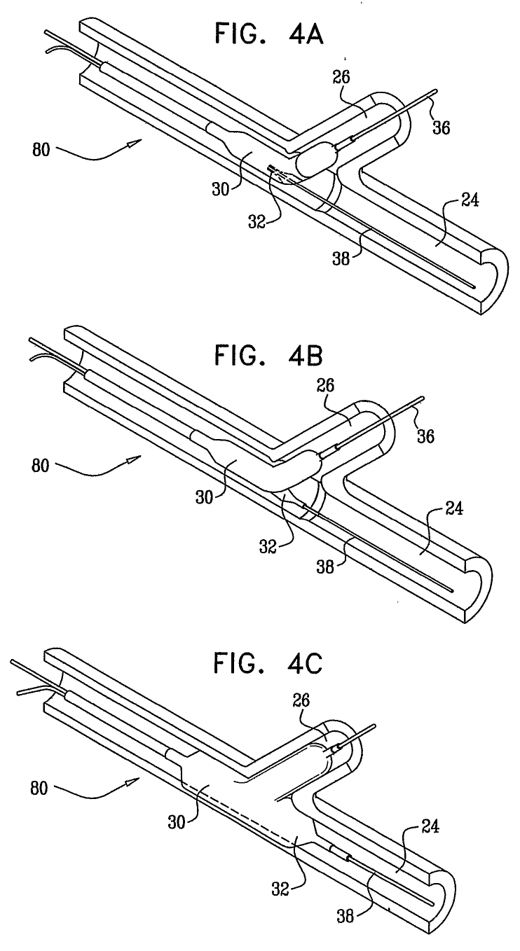

FIGS. 4A-4C are pictorial illustrations of a system 80 for treating a vascular bifurcation, in accordance with an embodiment of the present invention. System 80 provides balloon 30 for treatment of the bifurcation, but without a stent. Operation of balloon 30 in the context of system 80 is otherwise similar to the manner described above with respect to system 20 (FIGS. 1A-1C).

FIG. 4A is an illustration of balloon 30 inserted through main vessel 24 along guidewires 36 and 38, according to the operations described above with respect to FIG. 1A. Guidewire 36 extends through the length of balloon 30 and leads from the main vessel into side vessel 26. Guidewire 38 leads through main vessel 24 past the bifurcation, while exiting balloon 30 through protrusion 32. Before expansion, protrusion 32 may be folded into the main body of balloon 30, or held compactly against the side of the main body. Various configurations for an unexpanded protrusion of a catheter balloon are described in the abovementioned PCT Patent Publication WO 2005/041810 A2.

While uninflated, balloon 30 is moved into side vessel 26 along guidewire 36, thereby causing balloon 30 to bend. The bend of balloon 30 aligns protrusion 32, at the exit point of guidewire 38, along the axis of main vessel 24.

FIG. 4B is an illustration of an initial stage of inflation of balloon 30, after insertion of balloon 30 into side vessel 26. As indicated in the illustration, protrusion 32 begins to extend from a section of a side of balloon 30, in the distal direction of main vessel 24 past the bifurcation. Inflation of balloon 30 typically continues until the main body of the balloon and the protrusion are expanded to fill the vasculature of the bifurcation, as shown in FIG. 4C. The inflated, main body of balloon 30, as described above, has a curved shape. The curved shape may be customized to match the angle at which side vessel 26 branches away from main vessel 24.

Although the embodiments described above relate mainly to implantation of certain types of stents and balloons for vascular treatments, the principles of the stents and balloons used in these embodiments may similarly be applied to catheters of other types, such as catheters configured with self-expanding stents, as well as to balloon-based treatments of additional bodily orifices. It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A stent for treatment of a vascular bifurcation, comprising a distal member and a proximal member, the distal member and the proximal member comprising tubular bodies, the distal member comprising first struts oriented in the proximal direction and the proximal member comprising second struts oriented in the distal direction, wherein at least one of the first struts is connected to at least one of the second struts on a first side of the stent, while on a second side of the stent, opposite the first side, one or more of the first struts are unconnected to any of the second struts and one or more of the second struts are unconnected to any of the first struts.

2. The stent according to claim 1, wherein the unconnected first and second struts overlap while the stent is in an unexpanded, linear shape.

3. The stent according to claim 1, wherein the first side comprises a joint and wherein the stent is operative to bend along the joint, away from the second side, when the stent is moved from a main vessel into a side vessel at the vascular bifurcation.

4. The stent according to claim 3, wherein the unconnected first struts comprise a constriction of the unconnected second struts, and wherein the unconnected second struts are forced by the constriction to bend towards the first side responsively to the stent being moved into the side vessel.

5. The stent according to claim 4, wherein the unconnected first struts are operative to release the constriction of the unconnected second struts responsively to further bending of the stent.

6. The stent according to claim 4, wherein the unconnected first struts are operative to release the constriction of the unconnected second struts responsively to an expansion of the stent.

7. The stent according to claim 3, wherein responsively to being moved into the side vessel the stent is operative to perform an expansion, and wherein responsively to the expansion the unconnected second struts support a wall of the main vessel opposite the side vessel and the unconnected first struts support a wall of the main vessel distal to the bifurcation.

8. The stent according to claim 7, wherein the stent is operative to expand by a balloon that comprises a main body and a protrusion, the main body being shaped when expanded to bend within the bifurcation, and the protrusion being shaped when expanded to protrude along the axis of the main vessel and to extend into a portion of the main vessel distal to the bifurcation.

9. The stent according to claim 8, wherein the protrusion is operative to bend the unconnected first struts against the wall of the main vessel distal to the bifurcation.

10. A method for manufacturing a stent for treatment of a vascular bifurcation, comprising:
providing distal and proximal members comprising tubular bodies, the distal member comprising first struts oriented in the proximal direction and the proximal member comprising second struts oriented in the distal direction; and
connecting at least one of the first struts to at least one of the second struts on a first side of the stent, while not connecting one or more of the first struts on a second side of the stent, opposite the first side, to any of the second struts and not connecting one or more of the second struts on the second side to any of the first struts.

11. The method according to claim 9, and comprising overlapping the one or more of the unconnected first struts on the second side and the one or more of the unconnected second struts on the second side.

12. A method for treatment of a vascular bifurcation having a side vessel branching from a main vessel, the method comprising:
introducing into the main vessel in proximity to the side vessel a stent comprising a distal member and a proximal member, the distal member and the proximal member comprising tubular bodies, the distal member comprising first struts oriented in the proximal direction and the proximal member comprising second struts oriented in the distal direction, wherein at least one of the first struts is connected to at least one of the second struts on a first side of the stent, while on a second side of the stent, opposite the first side, one or more of the first struts are unconnected to any of the second struts and one or more of the second struts are unconnected to any of the first struts;
advancing the stent into the side vessel, such that the first side of the stent is bent with the distal member partially within the side vessel, and the second side of the stent faces away from the side vessel;
expanding the stent to support portions of the main vessel and the side vessel; and
bending the unconnected first struts to support a further portion of the main vessel distal to the vascular bifurcation.

13. The method according to claim 12, wherein supporting the further portion of the main vessel distal to the vascular bifurcation comprises supporting a portion of an ostium of the side vessel.

14. The method according to claim 12, and comprising carrying the stent on a balloon having a main body and a protrusion, wherein expanding the stent comprises expanding the main body to an expanded shape that bends within the bifurcation, and wherein bending the unconnected first struts comprises expanding the protrusion along the axis of the main vessel distal to the bifurcation.

15. A method for treatment of a vascular bifurcation having a side vessel branching away from an axis of a main vessel, the method comprising:
introducing into the main vessel in proximity to the side vessel a balloon having a main body and a protrusion;
advancing the main body partially into the side vessel such that the protrusion faces into a portion of the main vessel distal to the bifurcation;
expanding the main body, causing the main body to assume an expanded shape that bends within the bifurcation; and
expanding the protrusion, causing the protrusion to extend along the axis of the main vessel into a portion of the main vessel distal to the bifurcation.

16. The method according to claim 15, and comprising carrying a stent on the main body.

17. The method according to claim 16, wherein the stent comprises a distal member and a proximal member, the distal and proximal members comprising tubular bodies, the distal member comprising first struts oriented in the proximal direction and the proximal member comprising second struts oriented in the distal direction, wherein at least one of the first struts is connected to at least one of the second struts on a first side of the stent, while on a second side of the stent, opposite the first side, one or more of the first struts are unconnected to any of the second struts and one or more of the second struts are unconnected to any of the first struts, and
wherein carrying the stent comprises orienting the protrusion to face the second side of the stent,
wherein advancing the main body partially into the side vessel comprises advancing the stent into the side vessel, such that the first side of the stent is bent, with the distal member partially within the side vessel and the second side facing away from the side vessel,
wherein expanding the main body comprises expanding the stent to support portions of the main vessel and the side vessel, and
wherein expanding the protrusion comprises bending the unconnected first struts against a wall of the main vessel distal to the bifurcation.
